(19) United States
(12) Patent Application Publication
(10) Pub. No.: US 2008/0249454 A1
(43) Pub. Date: Oct. 9, 2008

(54) COMPRESSION SOCK

(76) Inventor: Karl Mills, Unionville, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/061,199

(22) Filed: Apr. 2, 2008

Related U.S. Application Data
(60) Provisional application No. 60/909,784, filed on Apr. 3, 2007.

(51) Int. Cl. A61F 13/06; A41B 11/02

(52) U.S. Cl. 602/63; 2/240

(57) ABSTRACT

A novel stocking is disclosed having an advantageous combination of static and graduated compression regions to provide beneficial circulation for individuals having compromised circulation and for athletes. Because the stockings compression is graduated to generally comply with muscle contours, the stockings can be easily placed on the foot and removed and substantially improve the circulation of those with a variety of circulatory problems. During exercise, improved circulation facilitates the removal of metabolites such as carbon dioxide and lactic acid from muscle tissue in the leg and increases oxygenation of the same muscle tissue. The novel stockings are constructed from a novel knit fabric comprising a taut elastic material providing sufficient stretch to allow the stockings to be easily placed on a foot. The elastic material is further responsible for static and/or graduated compression in selected regions of a stocking. The second material can be elastic or non-elastic and is incorporated in to the knit material in a relaxed state to allow movement in a stretching direction without the second material being substantially stretched. A method for producing the novel stocking is also provided.

### BACKGROUND

The present invention relates to elastic garments, and more particularly to compression stockings. Compression stockings have been widely used to improve athletic performance and to minimize the risk of embolism in individuals confined to a bed following surgery and for other reasons and for individuals suffering from chronic venous insufficiency (CVI). Compression stockings benefit each of these individuals by improving venous blood flow from the foot and lower leg regions back to the heart. When a patient is confined to bed, for example, after an operation, the likelihood of thrombosis is markedly increased due to a decrease in the velocity of blood flow in the patient's legs during confinement. Similarly, individuals suffering from CVI often have pooling of blood in the calf and ankle regions accompanied by discomfort. The typical graduated compression stockings cause application of a compressive pressure against the patient's leg which gradually decreases in a continuous manner from the lower ankle region toward the upper part of the leg. Such stockings increase the velocity of blood flow in the legs, and minimize the possibility of thromboembolism and/or the pooling of blood in the lower leg region.

During the strenuous exertion resulting from a variety of athletic performances, an athlete can experience a condition that approaches the condition of an individual suffering from CVI. As a result of the slower return of blood to the heart, blood can build up in the lower leg region resulting in reduced oxygenation of muscle tissue and a buildup in metabolic by-products such as lactic acid. Under these conditions, the muscle signals its condition with pain and reduced performance.

For graduated compression stockings to provide a benefit to the wearer, the stockings should be easy to place on the wearer, comfortable, and the stocking should provide sufficient compression to improve venous circulation without reducing circulation by overly compressing the lower leg. Compression stockings currently available are generally more difficult for the wearer to place on their foot, ankle and calf than non-compression stockings and unless properly fitted can result in discomfort and reduced circulation. In addition, because the angle of compression does not generally correspond to the shape of the leg muscles, an optimum compression is not provided. Although graduated compression stockings have been shown to benefit athletes and individuals confined to bed or suffering from CVI, compression stockings currently available have not addressed the problems described above. A compression stocking is needed which can be easily placed on the wearer and removed, which is comfortable, which can provide the circulatory benefits of compression stockings for an athlete, bed-fast patient, a sufferer of CVI, and the like, without a risk of reduced circulation caused by excessive compression. Finally, all of this should be available without requiring extensive fitting of the compression stocking.

### SUMMARY

A principal feature of the present disclosure is an improved compression stocking having a foot region, an ankle region, and a calf region. Compression stockings according to the current disclosure provide a graded compression which is maximum at or proximate the ankle region and which decreases along the course leading to the sock's upper region. However, unlike conventional compression stockings, the graded compression is not continuous but changes segments of the ankle and calf regions according to changes in general shape of the leg muscles. Each change results in a discontinuity in the socks angle of compression “ac”.

The angle of compression for a segment within a sock is defined as:

$$a c = \frac{- ( {c}_{2} - {c}_{1} )}{d}$$

where “ac” is the angle of compression, c₁ is the compression at a first edge of a segment and c₂ is the compression at the second edge of a segment, and d is the distance between the points where the compression measurements were taken. A negative sign is utilized to convert the negative slope generally obtained to a positive number. A segment having an angle of compression equal to zero (c₁=c₂) is a segment having static compression.

Socks prepared according to the present disclosure typically have at least an ankle region, and a calf region, with the two regions collectively having at least two segments capable of exerting a compressive force when the sock is worn. For socks provided according to the present disclosure, (i) the at least two segments have different angles of compression adapted to provide a different range of compressive forces over each segment; (ii) within each segment the compressive force is maximum proximate the ankle region or proximate a segment closest to the ankle region; and the ankle and calf regions are constructed from a material that can provide the desired level of compression, but remain easy to place on a wearer's foot.

A preferred material for preparing the improved compression stocking is constructed at least in part from a material having a taut elastic component and a relaxed component incorporated in a manner to provide static compressive forces, graduated compressive forces, or a combination of these forces within the stocking. In a typical embodiment, the elastic component remains in an un-stretched state in a stocking not worn and when placed on a wearer, stretches to provide a desired level of compression. The relaxed component, in the same embodiment exists in gathered state in a stocking not worn and expands without applying compression when placed on a wearer. As a result, substantially all of the compression generated by the stocking is derived from the elastic material's circumferential stretch. The foot, ankle and calf regions can be divided into separate segments with each separate segment providing no compression, static compression, or graduated compression.

A further feature of the present disclosure is a method of improving venous haemodynamics by selecting an improved compression stocking described above and wearing the stocking while at rest, during an active period or a combination thereof. Preferred stockings described above can decrease the amount of blood remaining in the leg region, increase the ambulatory venous pressure, and increase the ejection fraction with a decrease of reflux of blood in the lower leg region. The effects provided prove beneficial to bed-ridden patients, individuals suffering from CVI and to athletes undergoing strenuous exercise.

Finally, a further feature of the present disclosure is a method for constructing the improved stocking. One aspect of the method includes the steps of selecting materials for constructing a stocking; selecting a stocking plan consistent with the stocking's intended use; selecting a design for the stocking's fabric to implement the stocking's plan, connecting sources of the selected materials to a commercial knitting machine; programming the knitting machine to construct the stocking according to the selected design and knitting the stocking according to the selected design and plan. The materials selected can include materials commonly used to manufacture stockings provided at least one material is an elastic material.

The stockings plan includes aspects such as whether the stocking is a tube or conventional stocking, whether the stocking has padded regions in the foot area, whether the stocking has an inner hydrophobic layer, which regions have static compression and which regions have graduated compression and the like. Options for segments include segments having substantially no compression, segments having static compression and segments having graduated compression. The stocking's plan is generally determined by the stocking's intended use. The stocking's design typically refers to the manner in which the materials are selected, the path taken by the elastic material in passing through the wales of relaxed material, how the tautness of the elastic material is controlled to provide a static compression region or a graduated compression region, how the level of relaxation within the wales of relaxed material is varied to allow a region to expand without stretching the relaxed material, and the like. The stocking's design is determined by the stocking's plan. Further features will become more fully apparent in the following description of the embodiments of this disclosure.

### DESCRIPTION OF THE DRAWINGS

In the drawings:

FIG. 1A is a perspective view of one embodiment of the stocking of the present invention;

FIG. 1B is a plot of compression by segments of an embodiment illustrated in FIG. 1A;

FIG. 2A is a perspective view of another embodiment of the stocking of the present invention having an inner hydrophobic layer;

FIG. 2B is a plot of compression by segments of an embodiment illustrated in FIG. 2A;

FIG. 3A is a perspective view illustrating the stocking of FIG. 1 having padded segments in the foot region;

FIG. 3B is a plot of compression by segments of an embodiment illustrated in FIG. 3A; and

FIG. 4 is a stitch diagram for fabric having taut elastic component and a relaxed component.

### DESCRIPTION

For the purposes of promoting an understanding of the principles of this disclosure, references will now be made to several embodiments and specific language will be used to describe the same. It will nevertheless be understood that no limitation of the scope of the disclosure is thereby intended, such alterations and further modifications and applications of the principles of the disclosure as described herein being contemplated as would normally occur to one skilled in the art to which the disclosure relates.

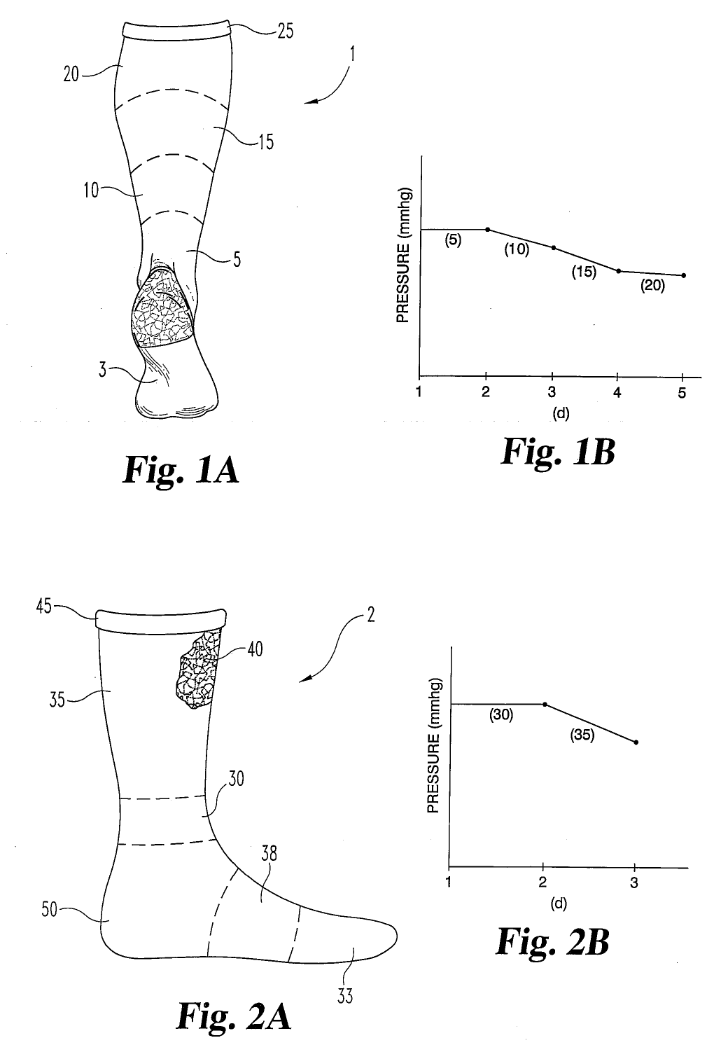

Referring now to FIG. 1A, there is shown an embodiment of a compression stocking generally designated 1 having a lower first circumferential generally cylindrical segment 5, and second circumferential generally cylindrical segment 10, a third circumferential generally cylindrical segment 15, a fourth circumferential generally cylindrical segment 20, and finally a fifth circumferential generally cylindrical welt 25. As shown, the first segment 5, a static compression region, is placed on the lower leg generally in the ankle region. Compression at the top and bottom of segment 5 is substantially the same and the height of segment 5 can vary from one embodiment to another, but generally corresponds to a portion of the lower leg having a generally constant circumference. The second segment 10 exerts a compressive force which is graduated throughout the segment that generally conforms to the muscle groups from the ankle to the lower calf portion of a wearer's leg. This segment has a generally low angle of compression causing compression for this segment to be minimally higher near the ankle region and minimally lower at the upper portion of the segment. The third segment 15 similarly exerts a compressive force which is graduated throughout the segment. However, because this segment has a greater angle of compression in order to comply with the shape of muscle groups in that region of the leg, the compression gradient over the segment is generally greater than for segment 10. As a result the compression gradient over this segment is generally greater than over segment 10. Like segment 5, the fourth segment 20, exerts a static compression over this region of the leg. Finally, a welt 25 is placed the upper edge of stocking 1 to tie off and secure the yarn ends and provide a smooth upper surface. Stocking 1 illustrates a tube form of a compression stocking of the present disclosure. Stocking 1 is particularly suitable for use in athletic events. Stocking 1 is easy to place on wearer's foot and provides sufficient compression in the necessary regions to improve circulation and thereby improve athletic performance.

FIG. 1B illustrates the compression distribution for the embodiment described above showing the relative pressure measurements for segments 5, 10, 15, and 20. The slope a given segment provides the measure of the angle of compression. In this particular embodiment, segment 15 has the greatest angle of compression, and segments 5 and 20 provide static compression.

Referring now to FIG. 2A, there is shown an embodiment of a compression stocking generally designated 2 having a lower foot segment which includes a toe region 33, an arch region 38 and a heel region 50. Above heel region 50 is a first circumferential generally cylindrical segment 30, a second circumferential generally cylindrical segment 35, and finally a third circumferential generally cylindrical welt 45. As shown, the first segment 30, a static compression region, is to correspond to the lower leg generally in the ankle region. Compression at the top and bottom of segment 30 is substantially the same and the height of segment 30 can vary from one embodiment to another. The second segment 35 exerts a compressive force which is graduated throughout the segment to generally conform to the muscle groups from the ankle to the lower calf portion of a wearer's leg. A welt 45 is placed the upper edge of stocking 2 to tie off and secure the yarn ends and provide a smooth upper surface. FIG. 2B illustrates the compression distribution for the embodiment described above showing the relative pressure measurements for segments 30 and 35. The slope a given segment provides the measure of the angle of compression. In this particular embodiment, segment 35 provides a measure of graduated compression while segment 30 provides static compression.

The inner surface of stocking 2 is constructed of a hydrophobic material to facilitate the transfer of moisture from the stocking's inner surface to the stocking's outer surface and to minimize the trapping of moisture in contact with a wearer's skin. Segment 38 is a compression region to provide support for the foot's arch. Segment 38 can be a static compression region or a graduated compression to conform to the normal shape of a foot's arch. Toe segment 33, arch segment 38, and heel segment 40 can be static compression, graduated compression generally devoid of compression, or a combination thereof. Similarly, segments 33, 38 and 50 can be combined into a single segment having a uniform construction throughout ranging from static compression, graduated compression or generally devoid of compression. Stocking 2 illustrates a below-the-calf stocking. Stocking 2, because of its wicking layer 40, is particularly suitable for wearing during athletic performances.

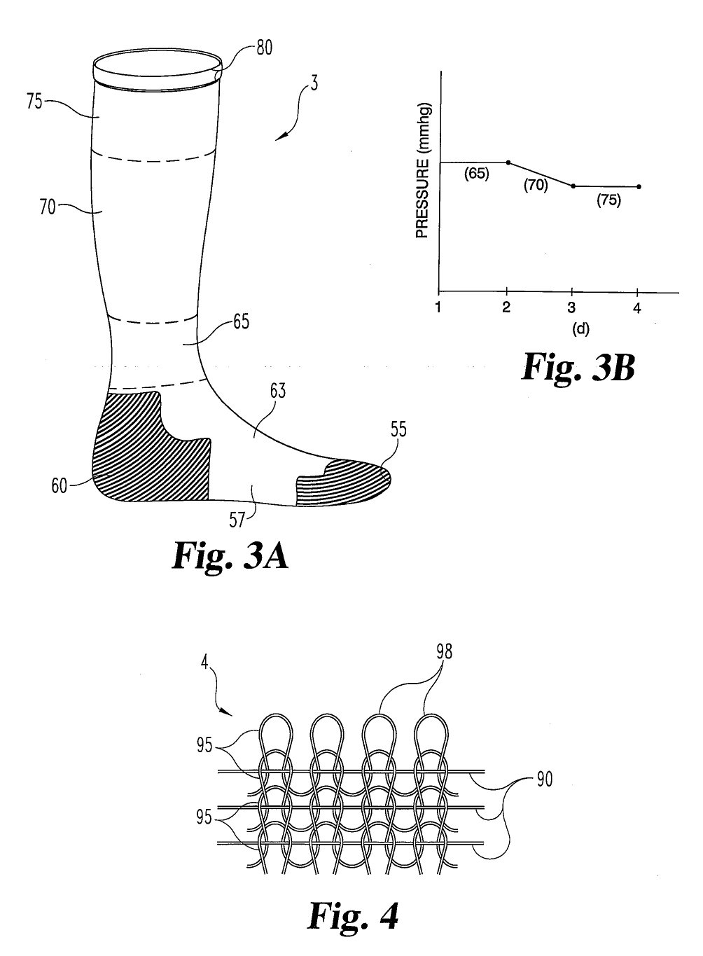

Referring now to FIG. 3A, there is shown an embodiment of a compression stocking generally designated 3 having a foot region 63, a lower first circumferential generally cylindrical segment 65, and second circumferential generally cylindrical segment 70, a third circumferential generally cylindrical segment 75 and finally a fifth circumferential generally cylindrical welt 80. As shown, the first segment 65, a static compression region, is placed on the lower leg generally in the ankle region. Compression at the top and bottom of segment 65 is substantially the same and the height of segment 65 can vary from one embodiment to another. The second segment 70 exerts a compressive force which is graduated throughout the segment that generally conforms to the muscle groups in this region of a wearer's leg. The third segment 75 exerts a static compressive force which is graduated throughout the segment. Finally, a welt 80 is placed the upper edge of stocking 3 to tie off and secure the yarn ends and provide a smooth upper surface.

The foot region 63 of stocking 3 includes a toe region 55 and a heel region 60. Either or both of regions 55 and 60 can be padded to provide additional support for these regions of a wearer's foot subject to repeated contacts with a walking or running surface. Preferred embodiments of stocking 3 include padded regions 55 and 60. Stocking 3 illustrates an over-the-calf embodiment of the present disclosure. Stocking 3 is particularly suitable for medical use and for athletic performance. The improved ease of placing stocking 3 onto a patient's foot is particularly important with regard to medical uses where a patient's willingness to wear a compression stocking can be compromised if the stocking is too difficult to place on the patient's foot. The padded regions 60 and 55 are particularly beneficial for patients suffering from diabetes.

FIG. 3B illustrates the compression distribution for the embodiment described above showing the relative pressure measurements for segments 65, 70 and 75. The slope a given segment provides the measure of the angle of compression. In this particular embodiment, segment 70 provides a measure of graduated compression while segments 65 and 75 provide static compression.

Referring now to FIG. 4, there is shown a preferred embodiment of a knitted fabric 4 having a taut elastic material 90 running perpendicular to the wales 98 constructed from a relaxed material 95. Knitted fabric 4 can provide a region with static compression provided the tension on elastic material 90 is relatively constant over the region providing static compression. Knitted fabric 4 can provide a region with graduated compression provided the tension is varied in a generally linear fashion across the region providing graduated compression. Fabric 4 is able to stretch along the direction of material 90 and provide a compressive force along this axis because of material 90's elastic properties. Fabric 4 is able to elongate to conform to along the direction of the elastomeric material 90 and at right angles to wale 98 because of the relaxed nature of material 95. For example, stocking 3 (See FIG. 3) having region 70 constructed of fabric 4 can be easily stretched when placed on a wearer's foot because material 90 allows a side to side stretching of region 70 and material 95 is sufficiently relaxed to allow the region to be expanded without requiring material 95 to stretch. Once in place stocking 3 having region 70 constructed from fabric 4 can provide a desired level of compression based on the elastic properties of material 90 and how taut it is in an un-stretched state. Further because material 95 has a relaxed condition, the stretching of region 70 can occur without the further stretching of material 95.

Further embodiments of fabric 4 can be constructed from an elastic material 90 having a generally taut and un-stretched condition and an elastic material 95, having a relaxed condition in the un-stretched condition. Such an embodiment has the advantages described above and provides even greater ability to stretch in all directions. A stocking constructed from this particular embodiment of fabric 4 would offer even greater ease of placing on an individual's foot while still controlling compression through material 90.

Material 90 can include but is not limited to an elastic material or yarn made of rubber, spandex or other elastic material such as Lycra® fiber. Such elastic materials and/or yarns can further include a covering of straight and/or textured filament yarns such as nylon, polyester or polypropylene. Lycra is a registered trademark of Investa North America S.A.R.L. Corporation Luxembourg, 4417 Lancaster Pike Legal—CRP 722/1032, Wilmington, Del. 19805.

Material 95 can include, but is not limited to synthetic fibers, natural fibers, and fibers derived from natural products. Synthetic fibers include but are not limited to nylon fibers, acrylic fibers, polyester fibers, and polypropylene fibers. Typical synthetic fibers are sold under the trade names Coolmax, Sensura and Comfortrel. Yarns having a natural source can be obtained from cotton, wool, bamboo, hemp, alpaca and the like. Yarns derived from and/or manufactured from a natural source can be obtained from soy protein, corn, and the like. Yarns having filament can have either a straight or textured form. Examples of such filament forms of yarn include, but are not limited to nylon, polyester, polypropylene and the like. The various yarns described herein can be used individually or in combination with each other. Further, the yarn combinations can be formed in the knitting process or in a separate process prior to the knitting process. Coolmax is a registered trademark of Investa North America S.A.R.L. Corporation Luxembourg, 4417 Lancaster Pike Legal—CRP 722/1032, Wilmington, Del. 19805. Sensura is a registered trademark of Wellman, Inc., 1040 Broad Street, Suite 302, Shrewsbury, N.J. 07702. Comfortrel is a registered trademark of Fiber Industries, Inc. 1040 Broad Street, Suite 302, Shrewsbury, N.J. 07702.

Materials for constructing the hydrophobic layer 40 (See FIG. 2) include, but are not limited to materials having hydrophobic properties. Such hydrophobic yarns are typically formed by extrusion of hydrophobic polymers such as for example, polypropylene.

Methods for making stockings according to the present disclosure include both warp and weft knitting. Weft knitting is preferred because it is suited for industrial knitting machines. A person skilled in the art of operating commercial knitting machines and in possession of this present disclosure could program such a machine to knit the stockings of this present disclosure without undue experimentation. Similarly, a person experienced with hand knitting could also produce the stockings of this present disclosure without undue experimentation.

While applicant's disclosure has been provided with reference to specific embodiments above, it will be understood that modifications and alterations in the embodiments disclosed may be made by those practiced in the art without departing from the spirit and scope of the invention. All such modifications and alterations are intended to be covered.

What is claimed is:

1. A sock comprising an ankle region and a calf region; the ankle and calf regions collectively having at least two segments, each segment capable of exerting a compressive force when the sock is worn, and each segment having:
(a) a first compressive force “c₁” and a second compressive force “c₂”, separated by a distance “d”;
(b) an angle of compression [ac] defined by the equation
$$a c = \frac{- ( {c}_{2} - {c}_{1} )}{d}$$
wherein: (i) the at least two segments have different angles of compression adapted to provide different range of compressive forces over each segment, (ii) within each segment, the compressive force is maximum proximate the ankle region, and (iii) the ankle and calf regions are constructed from a material having a first taut elastic component and a second relaxed component, adapted to provide a one way circumferential stretch.

2. The sock of claim 1, wherein the angle of compression for the ankle region “ac¹” is zero, providing static compression to that region and the angle of compression for the calf region is greater than zero.

3. The sock of claim 2, further having an interior hydrophobic wicking layer.

4. The sock of claim 1, wherein the calf region further comprises a first calf segment having an angle of compression “ac²” and a second calf segment having an angle of compression “ac³”, the first calf segment, proximate the ankle region has an angle of compression greater than zero, and ac² is greater than ac³.

5. The sock of claim 4, wherein ac² is zero, providing static compression for the second calf segment.

6. The sock of claim 4, wherein ac³ is greater than zero and less than ac², providing graduated compression for the second calf segment.

7. The sock of claim 4, wherein the sock is a knee sock.

8. The sock of claim 4, wherein the sock is a tube sock.

9. The sock of claim 4, further having an interior hydrophobic wicking layer.

10. The sock of claim 1, wherein: (a) the calf region comprises a first calf segment proximate the ankle region and having an angle of compression “ac²”, a second calf segment proximate the first calf segment and having an angle of compression “ac³”, and a third calf segment proximate the second calf segment and having an angle of compression “ac⁴” and (b) ac³>ac² and ac⁴>ac¹.

11. The sock of claim 10, wherein the sock is a knee sock.

12. The sock of claim 10, wherein the sock is a tube sock.

13. The sock of claim 10, further having an interior hydrophobic wicking layer.

14. The sock of claim 1, wherein: (a) the calf region comprises a first calf segment proximate the ankle region and having an angle of compression “ac²”, a second calf segment proximate the first calf segment and having an angle of compression “ac³”, and a third calf segment proximate the second calf segment and having an angle of compression “ac⁴” and (b) ac³>ac² and ac⁴=ac¹=0.

15. The sock of claim 14, wherein the sock is a knee sock.

16. The sock of claim 14, wherein the sock is a tube sock.

17. The sock of claim 14, further having an interior hydrophobic wicking layer.

18. The sock of claim 1, wherein the relaxed component is an elastic component.

19. The sock of claim 1, wherein the relaxed component is a synthetic fiber.

20. The sock of claim 1, wherein the relaxed component is a natural fiber.

21. The sock of claim 1, wherein the relaxed component is a fiber derived from a natural product.
